(19) United States
(12) Patent Application Publication
(10) Pub. No.: US 2007/0066846 A1
(43) Pub. Date: Mar. 22, 2007

(54) Process for making (S)-Pregabalin

(76) Inventors: Asher Maymon, Petach Tikva (IL); Vinod Kumar Kansal, Faridabad (IN); Lilach Hedvati, Doar Na Hefer (IL)

Correspondence Address: KENYON & KENYON LLP, ONE BROADWAY, NEW YORK, NY, 10004, US

(21) Appl. No.: 11/402,625

(22) Filed: Apr. 11, 2006

Related U.S. Application Data
(60) Provisional application No. 60/670,425, filed on Apr. 11, 2005.

(51) Int. Cl. C07C 229/02

(52) U.S. Cl. 562/575; 548/537

(57) ABSTRACT

The invention encompasses processes for the synthesis of (S)-Pregabalin, (S)-(+)-3-(aminomethyl)-5-methylhexanoic acid.

### RELATED APPLICATIONS

This application claims the benefit of U.S. provisional application No. 60/670,425, filed Apr. 11, 2005; herein incorporated by reference.

### FIELD OF THE INVENTION

The present invention is directed to a process for the synthesis of (S)-Pregabalin, (S)-(+)-3-(aminomethyl)-5-methylhexanoic acid.

### BACKGROUND OF THE INVENTION

(S)-Pregabalin, (S)-(+)-3-(aminomethyl)-5-methylhexanoic acid, a compound having the chemical structure 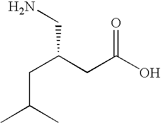
is also known as γ-amino butyric acid or (S)-3-isobutyl GABA. (S)-Pregabalin has been found to activate GAD (L-glutamic acid decarboxylase). (S)-Pregabalin has a dose dependent protective effect on-seizure, and is a CNS-active compound. (S)-Pregabalin is useful in anticonvulsant therapy, due to its activation of GAD, promoting the production of GABA, one of the brain's major inhibitory neurotransmitters, which is released at 30 percent of the brain's synapses. (S)-Pregabalin has analgesic, anticonvulsant, and anxiolytic activity.

(S)-Pregabalin may be prepared according to the process disclosed in U.S. Patent Application Publication No. 2003/0212290, by an asymmetric hydrogenation of a cyano-substituted olefin of formula 7, to produce a cyano precursor of (S)-3-(aminomethyl)-5-methyl hexanoic acid of formula 8, which is further reduced to obtain (S)-Pregabalin, as described in Scheme 1.

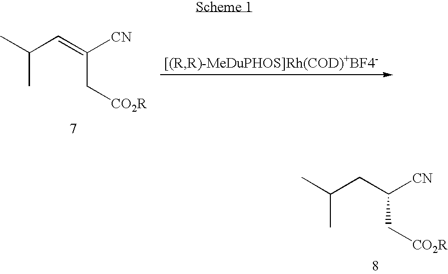

However, the disclosed method requires the use of carbon monoxide under high pressure, raising serious problems in adapting this process for production scale.

Another process is disclosed in JACS 2003, 125, 4442, in which an aluminum salen catalyst is used in the conjugate addition of hydrogen cyanide to α,β-unsaturated imides. 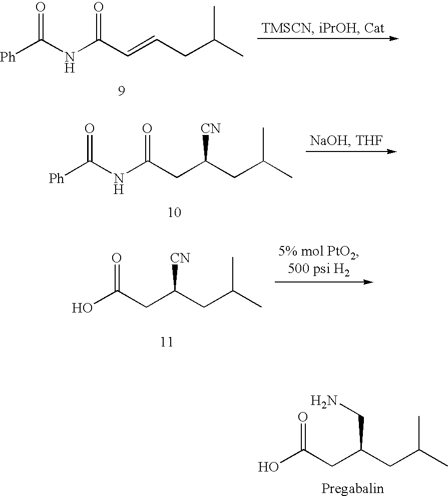
This process is also not practical for large scale production due to the use of highly poisonous reagents. In addition, the last reduction step requires high pressure of hydrogen, which only adds to the difficulties required for adapting this process for production scale.

Therefore, there is a need in the art for a process that overcomes these limitations.

### SUMMARY OF THE INVENTION

In one embodiment, the present invention provides the use of the compound of formula 15 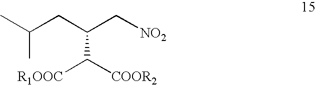
for the preparation of (S)-Pregabalin.

In another embodiment, the present invention provides the compound of formula 16, 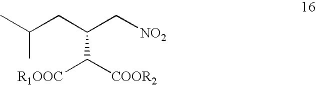
wherein R₁ and R₂ are independently H, a straight or branched C₁₋₁₀ alkyl, C₆₋₁₀ aryl, or C₃₋₆ allyl. Preferably, R₁ and R₂ each is methyl, ethyl, or isopropyl.

In yet another embodiment, the present invention provides the compound of formula 18, 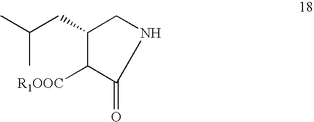
wherein, R₁ and R₂ are independently H, a straight or branched C₁₋₁₀ alkyl, C₆₋₁₀ aryl, or C₃₋₆ allyl. Preferably, R₁ and R₂ each is methyl, ethyl, or isopropyl.

In one embodiment, the present invention provides a process for the preparation of (S)-Pregabalin, denominated process 1, comprising combining the compound of formula 15, 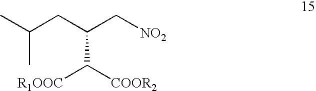
and a reducing agent; adding a copper salt and a solvent selected from a group consisting of: acetonitrile, toluene and mixtures of alcohol/acetonitrile; and heating, wherein R₁ and R₂ are independently H, a straight or branched C₁₋₁₀ alkyl, C₆₋₁₀ aryl, or C₃₋₆ allyl. Preferably, each of R₁ and R₂ is methyl, ethyl, or isopropyl.

In another embodiment, the present invention provides another process for the preparation of (S)-Pregabalin, denominated process 2, comprising combining the compound of formula 15 and a reducing agent; adding a salt, and a solvent selected from a group consisting of water, water miscible organic solvent and mixtures thereof; and heating.

In yet another embodiment, the present invention provides yet another a process for the preparation of (S)-Pregabalin, denominated process 3, comprising combining the compound of formula 15 a reducing agent, and a C₁₋₆ alcohol; combining with an inorganic acid to form a mixture; heating the mixture; and passing the mixture through an ion exchange resin.

In one embodiment, the present invention provides another process for the preparation of (S)-Pregabalin, denominated process 4, comprising combining the compound of formula 15, a salt, and a solvent selected from a group consisting of water, water miscible organic solvent and mixtures thereof; heating; adding a reducing agent; combining with an inorganic acid; heating, and passing through an ion exchange resin.

In another embodiment, the present invention provides another process for the preparation of (S)-Pregabalin, denominated process 5, comprising combining the compound of formula 15 a reducing agent, a Ni salt and a first solvent selected from a group consisting of: C₁₋₆ alcohol and THF; adding an inorganic base and a second C₁₋₆ alcohol; adding a C₆₋₁₀ aromatic hydrocarbon; heating; combining with an inorganic acid; heating; and mixing with a third C₁₋₆ alcohol and an organic base.

In yet another embodiment, the present invention provides a process for preparing pharmaceutical formulation comprising mixing (S)-Pregabalin, prepared according to the processes of the present invention, and a pharmaceutically acceptable carrier.

### DETAILED DESCRIPTION OF THE INVENTION

The process of the present invention provides a process for the preparation of (S)-Pregabalin that does not require an optical resolution step, and is also easy to conduct, efficient, and thus, can be easily adapted to larger scales.

The present invention provides the use of the compound of formula 15 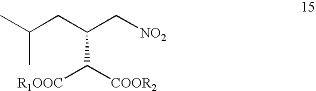
for the preparation of (S)-Pregabalin.

The present invention also provides the compound of formula 16, 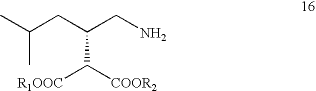
wherein R₁ and R₂ are independently H, a C₁₋₁₀ straight or branched alkyl, C₆₋₁₀ aryl, or C₃₋₆ allyl. Preferably, each of R₁ and R₂ is methyl, ethyl, or isopropyl.

The present invention further provides the compound of formula 18, 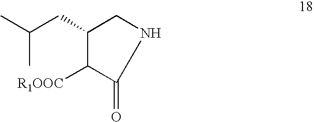
wherein preferably, R₁ and R₂ are independently H, a C₁₋₁₀ straight or branched alkyl, C₆₋₁₀ aryl, or C₃₋₆ allyl. Preferably, each of R₁ and R₂ is methyl, ethyl, or isopropyl.

The present invention provides a process for the preparation of (S)-Pregabalin, denominated process 1, comprising combining the compound of formula 15, 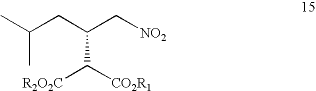
and a reducing agent; adding a copper salt and a solvent selected from a group consisting of: acetonitrile, toluene and mixtures of alcohol/acetonitrile; and heating, wherein each of R₁ and R₂ is independently H, a straight or branched C₁₋₁₀ alkyl, C₆₋₁₀ aryl, or C₃₋₆ allyl. Preferably, each of R₁ and R₂ is methyl, ethyl, or isopropyl.

Preferably, the process may be done according to the following scheme 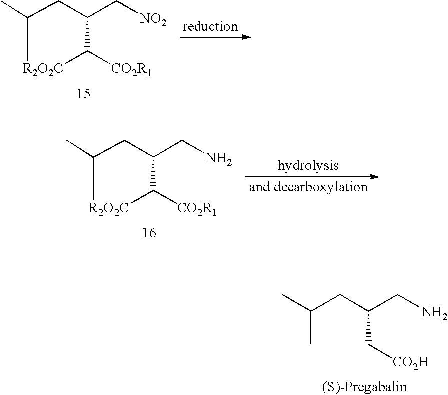
wherein R₁ and R₂ are as described above.

The compound of formula may be prepared, for example, according to the process disclosed in JACS, 2004, 126, 9906.

Preferably, the reduction step may be catalyzed by an acid; hence, an acid may be combined with the compound of formula 15 and a reducing agent. Preferably, the acid is an organic acid, more preferably, either acetic acid or formic acid. The acid may be used also as a solvent.

Preferably, the reducing agent is a combination of hydrogen and a catalyst. More preferably, the catalyst is a metal catalyst. The metal catalyst is selected from a group consisting of: Raney Ni, Pt and Rt. Preferably, the metal catalyst is palladium, and more preferably, palladium absorbed on carbon. Preferably, the hydrogen is bubbled at a pressure of about 1 to about 5 atmospheres, and more preferably, at a pressure of about 2 to about 5 atmospheres.

Preferably, combining the compound of formula 15, an acid and a reducing agent is performed at a temperature of about 15° C. to about 35° C., and more preferably, at about 25° C. to about 30° C., to provide a mixture. The mixture is maintained at the temperature for about 1 to about 10 hours, preferably, for about 2 to about 4 hours, and more preferably, for about 3 hours, to provide the compound of formula 16.

The compound of formula 16 may be recovered by filtering off the catalyst and evaporating the filtrate to obtain a residue.

Preferably, the copper salt is copper (I) salt, and more preferably, a copper oxide salt.

Preferably, adding the copper salt and a solvent selected from a group consisting of: acetonitrile, toluene and mixtures of alcohol/acetonitrile, provides a mixture, which is warmed at a temperature of about 60° C. to about 100° C., more preferably, of about 70° C. to about 90° C., and even more preferably, of about 80° C. The mixture is then maintained at the temperature for about 5 to about 10 hours, preferably, for about 7 to about 9 hours, and more preferably, for about 7.5 hours.

(S)-Pregabalin may be recovered by concentrating the maintained mixture, preferably, under vacuum, to obtain a residue. The residue may be purified by crystallization from a solvent selected from a group consisting of: mixtures of isopropyl alcohol and water, preferably, in a ratio of 65:30, of ethanol and water, of methanol and ethanol and of isopropanol and any other alcohol.

The present invention further provides another process, denominated process 2, for the preparation of (S)-Pregabalin comprising combining the compound of formula 15 and a reducing agent; adding a salt and a solvent selected from a group consisting of water, water miscible organic solvent and mixtures thereof; and heating.

Preferably, the process is done according to the above scheme, but with altering the reaction from compound 16 to (S)-Pregabalin.

Preferably, the salt is either an organic salt or an inorganic salt. Preferably the inorganic salt is an alkali salt. Preferably, the alkali salt is selected from a group consisting of: LiI, LiCl, NaCl, and KCN. Preferably, the organic salt is Bu₄NOAc. More preferably, the salt is an inorganic salt, most preferably, alkali salt, and even most preferably, NaCl.

Preferably, the water miscible organic solvent is selected from a group consisting of: dimethylsulfoxide (referred to as DMSO), N,N-dimethylformamide (referred to as DMF), dimethylacetamide (referred to as DMA), and hexamethylphosphoroustriamide (referred to as HMPT). The more preferred solvent is a mixture of water and DMSO.

Preferably, adding an alkali halide salt, a solvent selected from a group consisting of water, water miscible organic solvent and mixtures thereof provides a mixture, which is heated at a temperature of about 100° C. to about 160° C., preferably, of about 120° C. to about 140° C., more preferably, of about 135° C. The mixture is maintained at the temperature for about 4 to about 12 hours, preferably, for about 6 to about 8 hours, and more preferably, for about 7 hours.

(S)-Pregabalin may be recovered by cooling the maintained mixture, preferably, gradually. First the mixture is cooled at a temperature of about 30° C. to about 60° C., preferably, of about 35° C. to about 55° C., and more preferably, of about 40° C., and then to about 10° C. to about 0° C. Prior to the second cooling step, a solvent selected from a group consisting of diethylether, diisopropylether (referred to as DIPE) and t-butylmethylether (referred to as TBME) is added. After reaching a temperature of about 10° C. to about 0° C., water is added, and the mixture is further maintained at the temperature for about 25 minutes. The mixture separates into two phases and the aqueous phase is extracted with a solvent selected from a group consisting of: diethylether, DIPE and TBME, followed by washing the organic phase with water, and evaporating the solvent. (S)-Pregabalin may be purified by crystallization from a mixture of isopropyl alcohol (referred to as IPA) and water or from a mixture of tetrahydrofuran (referred to as THF) and water.

The present invention also provides a process for the preparation of (S)-Pregabalin, denominated process 3, comprising combining the compound of formnula 15 a reducing agent, and a C₁₋₆ alcohol; combining with an inorganic acid; heating; and passing through an ion exchange resign.

Preferably, the process is done according to the above scheme, but without isolating compound 16.

Preferably, the C₁₋₆ alcohol is ethanol.

Preferably, the reducing agent is a combination of hydrogen and a catalyst, and more preferably, a metal catalyst. The metal catalyst is selected from a group consisting of: Raney Ni, Pt and Rt. Preferably, the metal catalyst is Raney Nickel. Preferably, the hydrogen is bubbled at a pressure of about 1 to about 6 atmospheres, and more preferably, at a pressure of about 1 to about 3 atmospheres.

Preferably, combining the compound of formula 15, a C₁₋₆ alcohol and a reducing agent is done at a temperature of about 15° C. to about 40° C., and preferably, at about 25° C. to about 35° C., providing a mixture. The mixture is maintained at this temperature for about 3 to about 10 hours, preferably, for about 4 to about 6 hour, and more preferably, for about 5 hours, and then, preferably, a work-up step is done.

The work-up step is done by filtering off the catalyst and evaporating the filtrate to obtain a residue containing of compound of formula 16. The residue is then dissolved in the inorganic acid, and heated to a temperature of about 50° C. to about 100° C., preferably at about 80° C. to about 100° C., and more preferably, to about 100° C., for about 5 to about 20 hours, preferably, for about 10 to about 18 hours, and more preferably, for about 15 hours, to provide an inorganic acid salt of (S)-Pregabalin. The salt may be recovered by cooling the maintained mixture at a temperature of about 20° C. to about −10° C., and preferably, of about 10° C. to about 0° C., and evaporating water to dryness. Preferably, the inorganic acid is selected from a group consisting of: HCl, HBr, H₂SO₄ and H₃PO₄. More preferably, the inorganic acid is HCl. Preferably, the inorganic acid salt of (S)-Pregabalin is (S)-Pregabalin hydrochloride. The salt may be purified by slurry from a mixture of methanol and ether. (S)-Pregabalin hydrochloride may be converted to (S)-Pregabalin by passing it through an ion exchange resign, preferably, through Dowex 50W.

Optionally, the salt of (S)-Pregabalin may be converted to (S)-Pregabalin by dissolving it in isobutanol and adding an organic base, providing a mixture. The mixture is then maintained at a temperature of about 15° C. to about 55° C., preferably, of about 20° C. to about 35° C., for about 25 to about 80 minutes, preferably, for about 30 to about 55 minutes, and even more preferably, for about 45 minutes. (S)-Pregabalin may be recovered by filtering off the product, washing and drying. Preferably, the base is trialkylamine, more preferably, triisopropylamine, trimethylamine or triethylamine, most preferably, triethylamine.

The present invention provides another process for the preparation of (S)-Pregabalin, denominated process 4, comprising combining the compound of formula 15 a salt, and a solvent selected from a group consisting of water, water miscible organic solvent and mixtures thereof; heating; adding a reducing agent; combining with an inorganic acid; heating; and passing through an ion exchange resin.

Preferably, the process may be done according to the following scheme 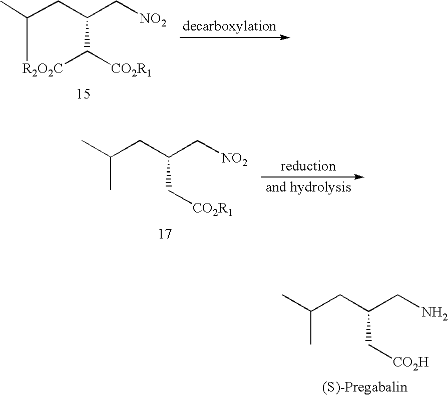
wherein R₁ and R₂ are described above.

The preferred salt, solvent and the inorganic acid are described a above.

Preferably, adding a salt, a solvent selected from a group consisting of water, water miscible organic solvent and mixtures thereof provides a mixture, which is heated at a temperature of about 145° C. to about 155° C. The mixture is maintained at the temperature, for about 3 to about 9 hours, preferably, for about 4 to about 6 hours, and more preferably, for about 5 hours, to provide the compound of formula 17.

The compound of formula 17 may be recovered by the same process as compound of formula 16 was recovered.

Preferably, the step from compound 17 to (S)-Pregabalin may be done by reducing the compound of formula 17 under the same conditions of the reduction of compound 15 to compound 16, as described in process No. 1, followed by obtaining the inorganic salt of (S)-Pregabalin, preferably, (S)-Pregabalin hydrochloride, which is then converted to (S)-Pregabalin. The inorganic salt of (S)-Pregabalin, preferably, (S)-Pregabalin hydrochloride, may be obtained by reacting the compound of formula 17 with an inorganic acid, preferably, HCl, under the same conditions of the reaction of compound of formula 16 with an inorganic acid, preferably, HCl, as described in process No. 3. The inorganic salt of (S)-Pregabalin, preferably, (S)-Pregabalin hydrochloride, may be converted to (S)-Pregabalin, by the methods disclosed in process No. 3, i.e. either by passing through an ion exchange resin, or by reacting with a base.

The present invention further provides another process for the preparation of (S)-Pregabalin, denominated process 5, comprising combining the compound of formula 15 a reducing agent, a Ni salt and a first solvent selected from a group consisting of: C₁₋₆ alcohol and THF; adding an inorganic base and a second C₁₋₆ alcohol; adding a C₆₋₁₀ aromatic hydrocarbon; heating; combining with an inorganic acid; heating, mixing with a third C₁₋₆ alcohol and an organic base,

Preferably, the process may be done according to the following scheme 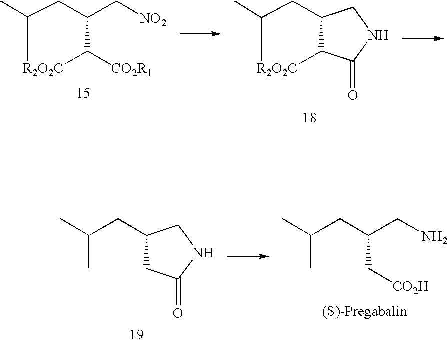
wherein each of R₁ and R₂ is as described above.

Preferably, the reducing agent is a metal hydride. Preferably, the metal hydride is selected from a group consisting of: sodium borohydride, sodium cyanoborohydride and lithium cyanoborohydride. More preferably, the metal hydride is sodium borohydride.

Preferably, the Ni salt is a Ni halide salt. The Ni halide is either NiBr₂ or NiCl₂ sesquihydrate. More preferably, the Ni halide is NiCl₂ sesquihydrate.

Preferably, the C₁₋₆ alcohol is selected from a group consisting of: methanol, ethanol, and IPA. More preferably, the first solvent is methanol.

Preferably, combining the compound of formula 15, a reducing agent, a Ni salt and a first solvent selected from a group consisting of: C₁₋₆ alcohol and THF is done at a temperature of about −10° C. to about 10° C., more preferably, at about 0° C. to about 5° C., and even more preferably, at about 0° C., providing a mixture. The mixture is then maintained at the temperature for about 3 to about 12 hours, preferably, for about 5 to about 8 hours, and more preferably, for about 6 hours, and quenched, providing compound 18; wherein R₂ is an alkyl group.

Preferably, quenching is done using NH₄Cl.

The compound of formula 18 may be recovered by adding a solvent selected from a group consisting of: CH₂Cl₂, toluene and dichloroethane, to the quenched mixture, and concentrating the organic phase.

Preferably, the inorganic base is an alkali hydroxide. Preferably, the alkali hydroxide is selected from a group consisting of: NaOH, KOH and LiOH. The preferred alkali hydroxide is NaOH.

Preferably, the second C₁₋₆ alcohol is selected from a group consisting of: methanol, ethanol, and IPA. More preferably, the C₁₋₆ alcohol is ethanol.

Preferably, adding an inorganic base and a second C₁₋₆ alcohol is done at a temperature of about 15° C. to about 55° C., preferably, at about 20° C. to about 35° C., providing a reaction mixture, which is maintained at the temperature for about 25 to about 90 minutes, preferably, for about 30 to about 60 minutes, and more preferably, for about 30 minutes, providing the compound of formula 18, wherein R₂ is H.

The compound of formula 18, wherein R₂ is H, may be recovered by concentrating the maintained reaction mixture, and adding water and an acid selected from a group consisting of: HCl, HBr, H₂SO₄, and H₃PO₄. Preferably, the acid is HCl. Subsequently, the phases are separated, and the aqueous phase is extracted with CH₂Cl₂. The combined organic phases are then concentrated.

Preferably, the C₆₋₁₀ aromatic hydrocarbon is either toluene or xylene.

Preferably, compound of formula 18, wherein R₂ is H, is dissolved in C₆₋₁₀ aromatic hydrocarbon. The solution is then heated at a temperature of about 90° C. to about 120° C., preferably, of about 100° C. to about 115° C., and more preferably, of about 110° C., and maintained for about 3 to about 12 hours, preferably, for about 6 to about 8 hours, and more preferably, for about 6 hours, providing compound 19.

The compound 19 may be recovered by concentrating the maintained mixture to dryness. Compound 19 may be purified by chromatography.

Preferably, the inorganic acid is selected from a group consisting of: HCl, HBr and H₂SO₄. More preferably, the inorganic acid is HCl.

Preferably, adding an inorganic acid provides a solution, which is warmed at a temperature of about 80° C. to about 105° C., preferably, to about 95° C. to about 100° C., and more preferably, to about 100° C., and maintained for about 10 to about 25 hours, preferably, for about 12 to about 18 hours, and more preferably, for about 15 hours, providing the inorganic salt of (S)-Pregabalin. The salt of (S)-Pregabalin is then converted to (S)-Pregabalin as described above.

The present invention also provides a process for preparing pharmaceutical formulation comprising mixing (S)-Pregabalin, prepared according to the processes of the present invention, and a pharmaceutically acceptable carrier.

### EXAMPLES

Having described the invention with reference to certain preferred embodiments, other embodiments will become apparent to one skilled in the art from consideration of the specification. The invention is further defined by reference to the following examples describing in detail the preparation of the composition and methods of use of the invention. It will be apparent to those skilled in the art that many modifications, both to materials and methods, may be practiced without departing from the scope of the invention.

### Example 1

### Preparation of, (S)-2-carboethoxy-5-methyl-3-nitromethyl hexanoic acid ethyl ester, Compound 16

A solution of 10 g of compound 15 in 150 ml of acetic acid is hydrogenated over a 10 percent palladium on carbon catalyst for 3 hours at ambient temperature and pressure, e.g., about 25° C. and about 1 to about 5 atmospheres pressure. The catalyst is then filtered off, and the filtrate is evaporated under reduced pressure giving compound 16.

### Example 2

### Preparation of (S)-Pregabalin from Compound 16

Method 1

First, 0.85 g of copper (I) oxide is added to a solution of 8.5 g of the dicarboxylic acid of compound 16 in 110 ml of CH₃CN. The resulting solution is warmed to 80° C., stirred for 7.5 hours, and then concentrated in vacuo. The residue is recrystallized from isopropyl alcohol/water in a 65:30 ratio, producing (S)-Pregabalin.

Method 2

First, 6 g of sodium chloride and 3 ml of water are added to a solution of 12 g of the diester compound 16 in 90 ml of DMSO. The mixture is heated to 135° C., and stirred for 7 hours. The mixture is then cooled to 40° C., and treated with 50 ml of methyl tert-butyl ether. The mixture is then cooled to 0° to 10° C., and 50 ml of water are added, while maintaining the temperature below 40° C. After stirring for 25 minutes, the phases are separated, and the aqueous phase is extracted with 35 ml of methyl tert-butyl ether. The organic extracts are combined, extracted with water, and then dried over sodium sulfate. After separation, the salt solution is concentrated in vacuo to dryness to provide crude (S)-Pregabalin. Crystallization from isopropyl alcohol/water in a 55:20 ratio provides the pure product.

Method 3:

### Preparation of (S)-Pregabalin Without Isolation of Compound 16

A mixture of 4 g of compound 15, 60 ml of ethanol, and Raney Ni is stirred at room temperature under an atmosphere of H₂ for 5 hours. The resulting mixture is filtered through a pad of Celite, and the filtrate is concentrated. The residue is then suspended in 40 ml of 6 N HCl, and the mixture is heated at 100° C. for 15 hours. After cooling, the excess water is removed under reduced pressure, producing a solid residue. Triturating the residue in methanol/ether provides the final product of (S)-Pregabalin hydrochloride. The crude product is purified by ion exchange chromatography on Dowex 50W to obtain (S)-Pregabalin. (S)-Pregabalin can be obtained also as described in example 7.

### Example 3

### Preparation of (S) 5-methyl-3-nitromethyl hexanoic acid ethyl ester, Compound 17

First, 7 g of sodium chloride and 5 ml of water are added to a solution of 9.6 g of the diester of compound 15 in 65 ml of DMSO. The mixture is heated to 145° to 155° C., and stirred for 5 hours. The mixture is then cooled to 40° C., and treated with 50 ml of methyl tert-butyl ether. The mixture is cooled to 0° to 10° C., and 25 ml of water are added, while maintaining the temperature at less than 40° C. After stirring for 25 minutes the phases are separated. The aqueous phase is extracted with 15 ml of methyl tert-butyl ether, the organic extracts are combined and extracted with 20 ml water, and dried over sodium sulfate. After separation, the salt solution is concentrated in vacuo to dryness to providing crude compound 17 as a yellowish oil.

### Example 4

### Preparation of (S)-Pregabalin from Compound 17

A solution of 10 g of 5-methyl-3-nitromethylhexanoic acid ethyl ester, compound 17, in 70 ml of acetic acid is hydrogenated over a catalyst of 10 percent palladium on carbon for 2.4 hours at ambient temperature and pressure, e.g., 25° C. and about 1 to about 5 atmospheres. The catalyst is then filtered off, the filtrate is evaporated under reduced pressure, and the residue is dissolved in 25 ml of 6 N HCl, followed by refluxing for 3 hours. The solution is evaporated under reduced pressure to dryness. The crude product is purified by ion exchange chromatography on Dowex 50W. Crystallization from isopropyl alcohol/water provides the pure product. It is important to note that first the initial product is the lactam, and the hydrolysis step provides the (S)-Pregabalin. In addition, this reduction can be performed with Raney nickel.

### Example 5

### Preparation of Compound 18

3.3 g of NaBH₄ is added to a suspension of 14 g of compound 15 and 5 g of NiCl₂.6H₂O in 140 ml of methyl alcohol at 0° C. The reaction mixture is stirred for 6 hours, and then quenched with NH₄Cl, followed by dilution with 55 ml of CH₂Cl₂. The organic phase is separated and dried over MgSO₄, filtered, and concentrated in vacuo to provide compound 18.

### Example 6

### Preparation of (S) 4-isobutylpyrrolidin-2-one, Compound 19

135 ml of 1 N NaOH is added to a solution of 24 g of compound 18 in 350 ml of ethanol at room temperature. After 30 minutes of stirring at that temperature, the reaction mixture is concentrated in vacuo. Then, 250 ml of 6 N HCl in water are added to the residue, and the phases are separated. The aqueous phase is extracted with 120 ML OF CH₂Cl₂, and then the combined organic layers are dried over MgSO₄, filtered, and evaporated under reduced pressure to provide the corresponding carboxylic acid (compound 18, wherein R₂ is H). A solution of the carboxylic acid in 120 ml of toluene refluxed at 140° C. for 6 hours, and then the mixture is concentrated under reduced pressure to dryness. The crude compound 19 is purified by column chromatography on silica gel to give desired pure compound 19.

### Example 7

### Preparation of (S) Pregabalin from Compound 19

10 g of compound 19 is dissolved in 440 ml 6 N HCl, and the solution is warmed to 125° C. for 15 hours. After cooling, the mixture is diluted with water, and extracted three times with dichloromethane, then the aqueous phase is evaporated. After drying under high vacuum, the (S)-Pregabalin hydrochloride is obtained as crystals. (S)-Pregabalin is further resolved by dissolving (S)-Pregabalin hydrochloride in isobutanol, and then adding triethyl amine. The mixture is stirred for 45 minutes, and the product is filtered, washed with isobutanol.

1. Compound 16 of the formula; 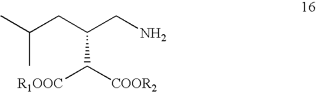
wherein R₁ and R₂ are independently selected from a group consisting of H, a straight or branched C₁ to C₁₀ alkyl, aryl, benzyl or substituted benzyl, and allyl.

2. Compound 18 of the formula. 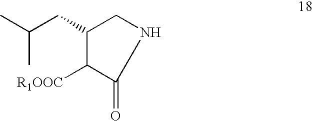
wherein R₁ and R₂ are independently selected from a group consisting of H, a straight or branched C₁ to C₁₀ alkyl, aryl, benzyl or substituted benzyl, and allyl.

3. The compound of any of the claims 1 and 2, wherein R₁ and R₂ are methyl, ethyl, or isopropyl.

4. A process for the preparation of (S)-Pregabalin, denominated process 1, comprising
a. combining the compound of formula 15, 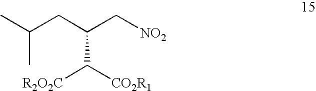
and a reducing agent;
b. adding a copper salt and a solvent selected from a group consisting of: acetonitrile, toluene and mixtures of alcohol/acetonitrile, and
c. heating.

5. A process for the preparation of (S)-Pregabalin, denominated process 2, comprising:
a. combining the compound of formula 15 and a reducing agent;
b. adding a salt, and a solvent selected from a group consisting of water, water miscible organic solvent and mixtures thereof; and
c. heating.

6. A process for the preparation of (S)-Pregabalin, denominated process 3, comprising:
a. combining the compound of formula 15 a reducing agent, and a C₁₋₆ alcohol;
b. combining with an inorganic acid;
c. heating; and
d. passing through an ion exchange resin.

7. A process for the preparation of (S)-Pregabalin, denominated process 4, comprising:
a. combining the compound of formula 15, a salt, and a solvent selected from a group consisting of water, water miscible organic solvent and mixtures thereof;
b. heating;
c. adding a reducing agent;
d. combining with an inorganic acid;
e. heating; and
f. passing through an ion exchange resin.

8. A process for the preparation of (S)-Pregabalin, denominated process 5, comprising:
a. combining the compound of formula 15, a reducing agent, a Ni salt, and a first solvent selected from a group consisting of: C₁₋₆ alcohol and THF;
b. adding an inorganic base and a second C₁₋₆ alcohol;
c. adding a C₆₋₁₀ aromatic hydrocarbon;
d. heating;
e. combining with an inorganic acid;
f. heating; and
g. mixing with a third C₁₋₆ alcohol and an organic base.

9. The process of any of the claims 4, 5, and 6, wherein the reaction is done according to the following scheme: 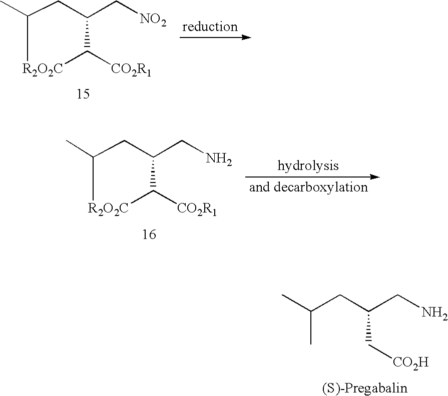
wherein R₁ and R₂ each independently is H, a straight or branched C₁₋₁₀ alkyl, C₆₋₁₀ aryl, or C₃₋₆ allyl.

10. The process of claim 7, wherein the reaction is done according to the following scheme: 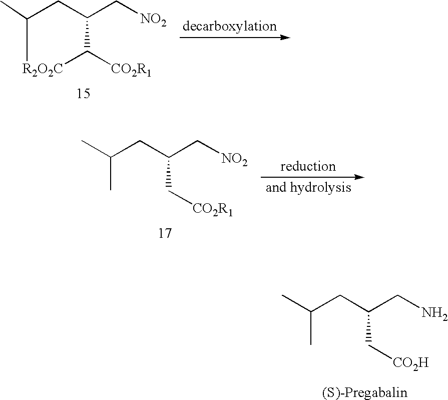
wherein R₁ and R₂ each is independently H, a straight or branched C₁₋₁₀ alkyl, C₆₋₁₀ aryl, or C₃₋₆ allyl.

11. The process of claim 7, wherein the reaction is done 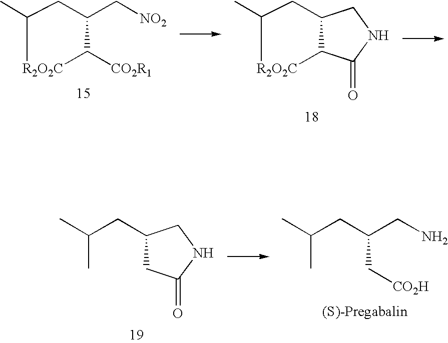
according to the following scheme:
wherein R₁ and R₂ each independently is H, a straight or branched C₁₋₁₀ alkyl, C₆₋₁₀ aryl, or C₃₋₆ allyl.

12. The process of claim 10, wherein R₁ and R₂ each is methyl, ethyl, or isopropyl.

13. The process of claim 7, wherein the reducing agent is catalyzed by an acid.

14. The process of any of the claims 4 and 5, further comprising adding an acid in step (a).

15. The process of any of the claims 14, wherein the acid is an organic acid.

16. The process of claim 15, wherein the organic acid is acetic acid.

17. The process of any of the claims 4, 5, and 7, wherein the acid is used also as a solvent.

18. The process of any of the claims 4, 5, 6, and 7, wherein the reducing agent is a combination of hydrogen and a catalyst.

19. The process of claim 18, wherein the catalyst is a metal catalyst.

20. The process of claim 19, wherein the metal catalyst is selected from a group consisting of: Raney Ni, Pd and Rt.

21. The process of claim 20, wherein the metal catalyst is either palladium or Raney Nickel.

22. The process of claim 20, wherein the palladium is absorbed on carbon.

23. The process of claim 18, wherein the metal catalyst is palladium absorbed on carbon, and the hydrogen is bubbled at a pressure of about 1 to about 5 atmospheres.

24. The process of claim 23, wherein the pressure is about 2 to about 5 atmospheres.

25. The process of claim 5, wherein the solvent in step (b) is acetonitrile.

26. The process of any of the claims 4 and 5, wherein step (a) is done at a temperature of about 15° C. to about 35° C.

27. The process of any of the claims 26, wherein the temperature is about 25° C. to about 30° C.

28. The process of any of the claims 4 and 5, wherein step (a) further comprises maintaining for about 1 to about 10 hours, prior to performing step (b).

29. The process of claim 28, wherein step (a) further comprises maintaining for about 2 to about 4 hours, prior to performing step (b).

30. The process of claim 4, wherein the copper salt is copper (I) salt.

31. The process of claim 30, wherein the copper salt is copper oxide.

32. The process of claim 4, wherein the heating in step (c) is done at a temperature of about 60° C. to about 100° C.

33. The process of claim 32, wherein the heating in step (c) is done at a temperature of about 70° C. to about 90° C.

34. The process of claim 4, wherein step (c) further comprises maintaining for about 5 to about 10 hours.

35. The process of any of the claims 5 and 7, wherein the salt is either an organic salt or an inorganic salt.

36. The process of claim 35, wherein the inorganic salt is an alkali salt.

37. The process of claim 36, wherein the alkali salt is selected from a group consisting of: LiI, LiCl, NaCl, and KCN.

38. The process of claim 37, wherein the alkali salt is NaCl.

39. The process of claim 35, wherein the organic salt is Bu₄NOAc.

40. The process of claim 35, wherein the salt is NaCl.

41. The process of any of the claims 5 and 7, wherein the water miscible organic solvent is selected from a group consisting of: DMSO, DMF, DMA and HMPT.

42. The process of claim 41, wherein the solvent is a mixture of water and DMSO.

43. The process of claim 5, wherein the heating in step (c) is done at a temperature of about 100° C. to about 160° C.

44. The process of claim 43, wherein the heating in step (c) is done at a temperature of about 120° C. to about 140° C.

45. The process of claim 5, wherein step (c) further comprises maintaining for about 4 to about 12 hours.

46. The process of claim 9, wherein compound 16 is not isolated.

47. The process of claim 6, wherein the C₁₋₆ alcohol is ethanol.

48. The process of claim 18, wherein the metal catalyst is Raney Nickel, and the hydrogen is bubbled at a pressure of about 1 to about 6 atmospheres.

49. The process of claim 48, wherein the hydrogen is bubbled at a pressure of about 1 to about 3 atmospheres.

50. The process of claim 6, wherein step (a) is done at a temperature of about 15° C. to about 40° C.

51. The process of claim 50, wherein step (a) is done at a temperature of about 25° C. to about 35° C.

52. The process of claim 6, wherein step (a) further comprises maintaining for about 3 to about 10 hours.

53. The process of claim 6, wherein the inorganic acid is selected from a group consisting of: HCl, HBr, H₂SO₄ and H₃PO₄.

54. The process of claim 53, wherein the inorganic acid is HCl.

55. The process of claim 6, wherein the heating in step (c) is done at a temperature of about 50° C. to about 100° C.

56. The process of claim 55, wherein the heating in step (c) is done at a temperature of about 80° C. to about 100° C.

57. The process of claim 6, wherein the heating in step (c) further comprises maintaining for about 5 to about 20 hours.

58. The process of any of the claims 6 and 7, wherein an inorganic salt of (S)-Pregabalin is obtained when combining with an inorganic acid and heating.

59. The process of claim 58, wherein the inorganic salt of (S)-Pregabalin is (S)-Pregabalin hydrochloride.

60. The process of any of the claims 6 and 7, wherein the last step of the process comprises converting the inorganic salt of (S)-Pregabalin to (S)-Pregabalin.

61. The process of any of the claims 6 and 7, further comprising converting the inorganic salt of (S)-Pregabalin to (S)-Pregabalin by:
a. dissolving the inorganic salt of (S)-Pregabalin in isobutanol;
b. adding an organic base, providing a mixture; and
c. maintaining the mixture at a temperature of about 15° C. to about 55° C.

62. The process of claim 61, wherein the organic base is trialkylamine.

63. The process of claim 62, wherein the trialkylamine is triisopropylamine, trimethylamine or triethylamine.

64. The process of claim 63, wherein the trialkylamine is triethylamine.

65. The process of claim 61, wherein step (c) is done for about 25 to about 80 minutes.

66. The process of claim 61, wherein step (c) is done at a temperature of about 20° C. to about 35° C.

67. The process of claim 7, wherein the heating in step (b) is done at a temperature of about 145° C. to about 155° C.

68. The process of claim 7, wherein step (b) further comprises maintaining for about 3 to about 9 hours.

69. The process of claim 7, wherein step (c) further comprises an acid.

70. The process of claim 7, wherein step (c) is done at a temperature of about 15° C. to about 35° C.

71. The process of claim 7, wherein step (c) further comprises maintaining for about 1 to about 10 hours prior to performing step (d).

72. The process of claim 6, wherein the heating in step (c) is done at a temperature of about 50° C. to about 100° C.

73. The process of claim 72, wherein the heating in step (c) is done at a temperature of about 80° C. to about 100° C.

74. The process of claim 73, wherein the heating in step (c) is done at a temperature of about 100° C.

75. The process of claim 6, wherein the heating in step (c) further comprises maintaining for about 5 to about 20 hours.

76. The process of claim 7, wherein the reducing agent is a metal hydride.

77. The process of claim 76, wherein the metal hydride is selected from a group consisting of: sodium borohydride, sodium cyanoborohydride and lithium cyanoborohydride.

78. The process of claim 77, wherein the metal hydride is sodium borohydride.

79. The process of claim 8, wherein the Ni salt is a Ni halide salt.

80. The process of claim 79, wherein the Ni halide is either NiBr₂ or NiCl₂ sesquihydrate.

81. The process of claim 80, wherein the Ni halide is NiCl₂ sesquihydrate.

82. The process of claim 8, wherein the first, second and third C₁₋₆ alcohol is selected from a group consisting of: methanol, ethanol, and IPA.

83. The process of claim 8, wherein the first C₁₋₆ alcohol is methanol.

84. The process of claim 8, wherein step (a) is done at a temperature of about −10° C. to about 10° C.

85. The process of claim 84, wherein step (a) is done at a temperature of about 0° C. to about 5° C.

86. The process of claim 7, wherein step (a) further comprises maintaining for about 3 to about 12 hours prior to performing step (b).

87. The process of claim 7, wherein in step (b) R₂ is an alkyl group in compound 18.

88. The process of claim 8, wherein the inorganic base is an alkali hydroxide.

89. The process of claim 88, wherein the alkali hydroxide is selected from a group consisting of: NaOH, KOH and LiOH.

90. The process of claim 89, wherein the alkali hydroxide is NaOH.

91. The process of claim 8, wherein the second C₁₋₆ alcohol is ethanol.

92. The process of claim 8, wherein step (c) is done at a temperature of about 15° C. to about 55° C.

93. The process of claim 92, wherein step (c) is done at a temperature of about 20° C. to about 35° C.

94. The process of claim 8, wherein step (c) further comprises maintaining for about 25 to about 90 minutes.

95. The process of claim 7, wherein, in step (c), R₂ is H in compound 18.

96. The process of claim 8, wherein the C₆₋₁₀ aromatic hydrocarbon is either toluene or xylene.

97. The process of claim 8, wherein the heating in step (f) is done at a temperature of about 90° C. to about 120° C.

98. The process of claim 8, wherein the heating in step (f) is done at a temperature of about 100° C. to about 115° C.

99. The process of claim 8, wherein step (f) further comprises maintaining for about 3 to about 12 hours.

100. The process of claim 8, wherein compound 19 is obtained in step (e).

101. The process of claim 8, wherein the inorganic acid is a strong acid.

102. The process of claim 100, wherein the inorganic acid is selected from a group consisting of: HCl, HBr and H₂SO₄.

103. The process of claim 102, wherein the inorganic acid is HCl.

104. The process of claim 8, wherein the heating in step (g) is done at a temperature of about 80° C. to about 105° C.

105. The process of claim 104, wherein the heating in step (g) is done at a temperature of about 95° C. to about 100° C.

106. The process of claim 8, wherein step (g) further comprises maintaining for about 10 to about 25 hours.

107. The process of claim 8, wherein (S)-Pregabalin hydrochloride is obtained in step (g).

108. The process of claim 8, wherein (S)-Pregabalin hydrochloride converted to (S)-Pregabalin in step (h).

109. A process for preparing pharmaceutical formulation comprising mixing (S)-Pregabalin, prepared according to any of the claims 4 to 8, and a pharmaceutically acceptable carrier.

110. The process of claim 9, wherein the reducing agent is catalyzed by an acid.

111. The process of claim 21, wherein the metal catalyst is palladium absorbed on carbon, and the hydrogen is bubbled at a pressure of about 1 to about 5 atmospheres.

112. The process of claim 22, wherein the metal catalyst is palladium absorbed on carbon, and the hydrogen is bubbled at a pressure of about 1 to about 5 atmospheres.

113. The process of claim 21, wherein the metal catalyst is Raney Nickel, and the hydrogen is bubbled at a pressure of about 1 to about 6 atmospheres.

114. The process of claim 60, further comprising converting the inorganic salt of (S)-Pregabalin to (S)-Pregabalin by:
a. dissolving the inorganic salt of (S)-Pregabalin in isobutanol;
b. adding an organic base, providing a mixture; and
c. maintaining the mixture at a temperature of about 15° C. to about 55° C.

115. The process of claim 114, wherein the organic base is trialkylamine.

116. The process of claim 115, wherein the trialkylamine is triisopropylamine, trimethylamine, or triethylamine.

117. The process of claim 116, wherein the trialkylamine is triethylamine.

118. The process of claim 117, wherein step (c) is done for about 25 to about 80 minutes.

119. The process of claim 118, wherein step (c) is done at a temperature of about 20° C. to about 35° C.
